(19) United States
(12) Patent Application Publication
(10) Pub. No.: US 2012/0129418 A1
(43) Pub. Date: May 24, 2012

(54) Silver coated nylon fibers and associated methods of manufacture and use

(76) Inventor: Edmund Michael Ingle, Winston-Salem, NC (US)

(21) Appl. No.: 13/360,700

(22) Filed: Jan. 28, 2012

Related U.S. Application Data
(63) Continuation-in-part of application No. 12/317,732, filed on Dec. 29, 2008.

(51) Int. Cl. B32B 15/14; B32B 27/36; B65D 30/08; A61L 2/00; A41D 13/00; A42B 3/00; A41D 13/08; A41D 13/06; B32B 5/18; A41D 27/26

(52) U.S. Cl. 442/371; 383/109; 2/459; 2/463; 2/467; 2/410; 2/16; 2/22; 422/5

(57) ABSTRACT

Silver coated nylon woven and non-woven fibers are disclosed that can be used to make fabrics, bags, and pads that are silver coated on one side of the fabric, bag, and/or pad. Silver coated nylon fabrics, bags, and pads provide an antimicrobial surface which remains on the surface and retains its antimicrobial characteristic until destroyed. The silver coating can be used as a way to eliminate, reduce and/or ameliorate odors emanating from the bacteria. The methods of adherence of the silver to the nylon may be performed in a number of ways. The pads may be further associated with protective gear that can be used by professions and athletes that use protective gear.

The present application claims priority under 35 USC §120 as a continuation in part application of U.S. application Ser. No. 12/317,732 filed Dec. 29, 2008, the entire contents of which are herein incorporated by reference in its entirety.

### FIELD OF THE INVENTION

The invention relates to methods of making and utilizing silver coated nylon fabrics. The invention also relates to overcoming the existing technological barriers for dyeing silver coated nylon fabrics. Prior silver coated nylon fabrics were 100% nylon and the surface was 100% silver coated following the fabric manufacturing process. Such fabrics are not suitable for end uses because of the critical obstacle of consumer desire for color in apparel. The fabrics currently available do not allow the application of color because 100% silver coated nylon fabric cannot hold dyes. Conversely, this invention involves creating silver coated nylon fabrics by incorporating the silver during or after the fabric making process, thereby adding color dyes to the non-silver plated fibers. Silver coated fabrics utilized in this invention use as an antimicrobial thermally conductive and electrically conductive fabric. The textile fabric, of the present invention, possesses a silver coating that remains on the surface of the fabric, retaining its characteristics for the life of the yarn. It is not subject to loss of effectiveness after a substantial number of standard washings and dryings. Accordingly, the invention encompasses silver coating of two sided, colored and multi fiber fabrics.

### DISCUSSION OF THE PRIOR ART

Silver kills hundreds of different types of bacteria and has been used for years in the medical field due to its antibacterial efficacy. There are many examples of present day medical products which utilize 100% silver coated nylon fabrics. These fabrics are used to treat burn victims and patients with open wounds to inhibit the growth of bacteria and kill any bacteria already in the wound. Silver has also been applied topically to fabrics as an antimicrobial agent but the process has lacked efficacy because the % of silver is generally less than 3%, the fabrics are not conductive because there is not a 100% coating of silver. These fabrics can be dyed but do not have the thermal conductivity or electrical conductivity of 100% silver plated fabrics. Other companies have added silver in the synthetic fiber manufacturing process, allowing the fibers to be dyed but the amount of silver in the polymer is generally less than 3%. These fibers are dyeable but are not thermally or electrically conductive and are not as effective in killing bacteria due to the low concentration of silver.

It would be extremely beneficial to generate effective permanent antimicrobial characteristics for film or textile surfaces, particularly on apparel fabrics, while maintaining the ability to add color to these fabrics.

In addition, silver-containing nylon fiber and fabrics are available commercially for electromagnetic shielding. Micro biocides have been adapted for incorporation within melt spun synthetic fibers, as taught within Japanese unexamined Patent Application No. H11-124729, which is herein incorporated by reference in its entirety. Although these fabrics can be dyed, they still suffer from drawbacks including the inability to conduct electricity, and ineffective antimicrobial activity.

Previously, attempts to apply these specific micro-biocides on the surfaces of fabrics and yarns have been unsuccessful. The dyeable fabrics as previously manufactured had a durability issue if treatment was topical and antimicrobial efficacy drawbacks (i.e., the kill rate above 99%/log kill rate ratio is over 2.0) if silver was added to the polymer prior to spinning. The topical approach of applying silver, with or without binders or the melt spinning of polymers with silver additives dosed in prior to spinning, generated fabric with a very low percentage of silver (or other equivalent biocidal metal). Thus, using the old methodology, the efficacy is much less than the electrolessly deposited silver coated nylon.

Topical treatment with silver or other metallic substrate has been successfully, applied to fabrics that without washing are somewhat durable. Although these silver-based agents provide relatively good, semi-durable, antimicrobial properties, they tend not to produce the desired long-lasting, wash-resistant, silver-based antimicrobial textile. Moreover, these fabrics tend to lack the thermal and electrical conductive properties of electrolessly silver plated fabrics. Silver coated nylon/non-nylon fabrics are desirable for textile applications, particularly if one wants the full biocidal strength of a silver coated nylon, yet needs the hand and color for normal textile uses. Such a combination fabric permits treatment of a fabric's individual fibers prior to or after, weaving and knitting, in order to provide greater versatility to the target yarn without altering its physical characteristics. Such a “combination” fabric must prove to be wash durable, particularly for apparel fabrics, in order to be functionally acceptable. Further, it is highly desirable for such a metalized treatment to be electrically conductive on the target fabric, yarn, and/or film surface. With the presence of metals and metal ions, a wash durable, antimicrobial, dyeable and electrically conductive treatment has not been previously available in the past. Such an improvement would thus provide an important advancement within the fabric and textile art. Although antimicrobial activity is one desired characteristic of the inventive metal-treated fabric, or film, this is not a required property of the inventive article. Odor-reduction, thermal conductivity, distinct colorations, reduced discolorations, improved yarn and/or fabric strength, resistance to sharp edges, etc., are all either individual or aggregate properties which may be accorded the user of such an inventive treated yarn, fabric, or film.

### DESCRIPTION OF THE INVENTION

This application discloses fabrics and other products which selectively exhibit antimicrobial characteristics. In one aspect of the invention, the fabric is comprised of two or more fibers, one of which is silver coated, and the other which is not silver coated. These fibers can be combined such that the silver coated fiber is predominantly on one side and the other non-silver coated fibers are on the reverse.

It is an object of the invention to provide a simple and effective manner of providing a coating on one side of a textile, fabric, or film with silver (or another metal) without coating the other side of the textile, fabric or film. This will allow the other side of the textile, fabric or film to be dyed or left in its natural state. A further object of the invention is to provide a textile, fabric or film that is electrically conductive and thermally conductive on one side so as to aid in the transportation and dissipation of heat and static build up while at the same time looking and feeling like a normal textile, fabric or film on the other side.

In one embodiment, the side of the textile, fabric, or film with the silver coating is nylon. One of the benefits of the present invention is that the use of nylon (and electrolessly plating the nylon side with silver) provides a textile, fabric or film that is far more durable than the corresponding topical treatment of textiles, fabrics or films with binders (which are subsequently treated with silver or another metal). In an embodiment, after plating, the fabric is not altered in terms of drape, and the non-silver plated fiber or fibers of the fabric still have the same hand before being treated

In addition, when the textile or fabric that has been electrolessly silver plated and cut and sewn into garments with the silver side of the fabric against the skin, wearers identify a positive effect on the wearer in terms of moving moisture off of the skin. This movement and evaporation of moisture from the skin is a result of the silver coated fibers reaching body temperature very rapidly due to the inherent thermal conductivity of silver. In addition, silver does not absorb moisture and unlike some fibers, such as cotton and wool, is not hydrophilic. The silver covers 100% of the surface of the nylon fibers and by accelerating the evaporation process, cools the wearer. Thus, the evaporation process is accelerated and the moisture that the wearer generates does not get absorbed by the silver coated nylon. The effect is pronounced when a two sided material such as un-plated polyester on the exterior of the garment (i.e., the side furthest away from the skin) is used. The exterior side of the fabric can aid in the wicking of the moisture across the fabric by capillary action and or surface treatments. In this embodiment, not only is there dissipation of heat from the wearer, but the wearer can safely wear an aesthetic garment that can be dyed or printed on the non-silver plated side, and worn with the printed or dyed fabric away from the skin. The fabric can be constructed in a manner that disguises the fact that there is a silver coated side to the garment.

Due to the nature of electrolessly plated nylon, the fabric can be washed in accordance with wash procedure AATCC test method 130-1981 for a plurality of washings and there is no measurable change in the surface of the silver coating. This demonstrates the ability of the textile, fabric, or film to perform in the same manner as when the product was first made. In one variation, the fabric can be washed 100 times without losing its effectiveness. Accordingly, in one embodiment, this invention encompasses a. textile, fabric, or film that has two or more components, with at least one of the components being made of nylon. The fabric is electrolessly plated, but only the nylon takes on the silver. The other components of the textile, fabric or film remain untreated. Thus, the textile, fabric, or film is comprised of individual fibers, with at least one of the fibers being nylon. The finish is adhered to at least one portion of the surface of the substrate and the electrolessly plated silver finish is integrally retained on the nylon surface of the substrate. Even after a plurality of washes as performed in accordance with the wash procedure of AATCC Test Method 130-1981, an amount of at least 65% of the amount of silver finish remains present on nylon portion of the surface of said substrate. The wash durability test noted above is standard and, as will be well appreciated by one of ordinary skill in this art, is not intended to be a required or limitation within this invention. Such a test method merely provides a standard which, upon 30 washes in accordance with such, the inventive treated substrate will not lose an appreciable amount of its electrically conductive metal treatment. In an embodiment, such wash durability will be maintained after 15 washes, after 20 washes, and alternatively 30 washes, all in accordance with the AATCC Test Method noted above.

Due to the multi-fiber component of the textile, fabric, or film, the combination of silver plated application to the nylon fibers does not exhibit any undesirable effects on the hand or drape of the target fabric itself. The metal finish is a solid finish on the nylon, such a finish which is noticeable by touch. Additionally, instrumental analysis can detect the actual conductivity of the fabric. In one embodiment an ohm meter can be used. The electrolessly silver plating enhances the feeling of the fabric and provides the desirable characteristics noted above.

Furthermore, the inventive dual purpose fabric, textile or film exhibits appreciable electrical conductivity (due to the 100% coating of the silver coated nylon fibers which are present throughout the target substrate) as measured by attaching a two-inch by two-inch fabric specimen to two electrodes and applying a voltage gradient of about 100 volts per inch through the fabric (i.e., in accordance with AATCC Test Method 76-1978). The measured resistance in ohms per square inch should be a minimum of 10 in order to provide a substantially electrically conductive fabric.

Nowhere within the prior art has such a specific treated dual purpose fabric, textile or film or method of making thereof been disclosed, utilized, or fairly suggested. Others have produced a product (marketed under the trade name X-STATIC®) which is a nylon yarn electrolessly plated with a silver coating on the 100% of the nylon yarn. This yarn is highly electrically conductive and can be utilized for static charge dissipation but cannot be dyed.

Any combinations of polyamides, such as nylon 6 and nylon 6.6, can be used with other fibers. Natural or synthetic fiber may be utilized as the base for coating the fabric, textile or film with silver as long as the other fiber can be dyed so the resulting substrate can hold color. Thus, natural (cotton, wool, and the like) or synthetic fibers (polyester, polyolefin, spandex and the like) may constitute one part of the target substrate, either by itself or in any combinations or mixtures. Synthetics, naturals, or blends or combinations thereof can be used. One embodiment uses at least 5% polyamides, such as nylon 6 and nylon 6.6, as the silver coated fiber. As for the other yarn types, for instance, and without intending any limitations therein, polyolefins, such as polyethylene, polypropylene, and polybutylene, halogenated polymers, such as polyvinyl chloride, polyesters, such as polyethylene terephthalate, polyester/polyethers, polyurethane, as well as spandex/elastane (block copolymer of polyurethane and polyethylene glycol) homopolymers, copolymers, or terpolymers in any combination of such monomers, and the like, may be utilized within this invention. In one embodiment polyethylene terephthalate (a polyester) can be used as the other fiber in the substrate.

Additionally, the target fabric may be coated with any number of different films, including those listed in greater detail below. As mentioned previously, the invention allows the non-silver coated fibers in the substrate to be dyed or colored to provide other aesthetic features for the end user with any type of colorant, such as, for example, poly(oxyalkylenated) colorants, as well as pigments, dyes, tints, and the like. Other additives may also be present on and/or within the target fabric or yarn, including antistatic agents, brightening compounds, nucleating agents, antioxidants, UV stabilizers, fillers, permanent press finishes, softeners, lubricants, curing accelerators, and the like. Other optional and supplemental finishes to the inventive fabrics can be employed such as soil release agents, which improve the wettability and washability of the fabric. Other finishes such as soil release agents may be employed and include those which provide hydrophilicity of the surface of polyester. With such a modified surface, again, the fabric imparts improved comfort to a wearer by wicking moisture. Additionally, other potential additives and/or finishes may include water repellent fluorocarbons and their derivatives.

In an embodiment treatment comprises at least 2%, alternatively 3%, or alternatively 5% of polyamide material such as nylon 6 or nylon 6.6 so that this component of the substrate can be electrolessly plated with metal. Silver provides good overall desired characteristics, such as, good antimicrobial and/or odor reducing characteristics, and, most importantly, wash durability on the target substrate as well as electrical and thermal conductivity. It is contemplated that other metals may also be suitable for their desired characteristics.

The term silver particle is intended to encompass any compound which comprises of silver in its elemental or ionic state (thus Ag° or Ag+ may be present). Metal salts may also be present in some amount either in a pure state, or reduced to produce the desired metal particles.

The selected substrate can be a combination of a polyamide yarn such as nylon 6 or nylon 6.6 and other fibers, or a fabric comprising individual fibers or yarns (with the ability of the polyamide fibers to be electrolessly silver plated). The non-polyamide individual fibers or yarns may be of any typical source for utilization within fabrics, including natural fibers (cotton, wool, ramie, hemp, linen, and the like), synthetic fibers (polyolefins, polyesters, polyaramids, acetates, rayon, acrylics, spandex/elastane and the like), and inorganic fibers (fiberglass, boron fibers, and the like). The target yarn may be of any denier, may be of multi- or mono-filament, may be false-twisted or twisted, or may incorporate multiple denier fibers or filaments into one single yarn through twisting, melting, spun and the like. The target fabrics may be produced of the same types of yarns discussed above, including any blends thereof. Such fabrics may be of any standard construction, including knit, woven, or non-woven forms.

The inventive fabrics may also be utilized in any suitable application, including, without limitation, apparel, upholstery, bedding, wiping cloths, towels, gloves, rugs, floor mats, drapery, napery, bar runners, textile bags, awnings, vehicle covers, boat covers, tents, and any similar applications.

The polyamide/other fiber substrates can be electrolessly silver plated either before or after the fabric is dyed.

In an embodiment, the products described herein that contain silver on the surface allow the release of silver ions upon activation with moisture so that they kill bacteria, thereby eliminating, reducing and/or ameliorating smell from the bacteria.

Accordingly, in the following embodiments, the products containing silver can be used to eliminate, reduce and/or ameliorate the smell that results from bacteria. In an embodiment, the odor is reduced and/or ameliorated. In an embodiment, silver is present on at least one side of the following products.

In an embodiment, the present invention relates to a bag that is waterproof with an antimicrobial inner lining of silver-coated poly/nylon non-woven fabric. In an embodiment, the bag is colored (e.g., dyed) on at least one side. The colors may be bright colors or muted colors or the bag may be printed. When a garment or other product that has bacteria associated with it is placed in the bag and the bag is shut, no smell emanates from the bag because the silver containing side (i.e., the inner side of the bag) kills any bacteria that contacts the inner lining thus eliminating, reducing and/or ameliorating any odor that may result from the bacteria. Moreover, in an embodiment, when a zipper is present on the bag, no odor emanates from the bag when the bag is closed and the silver lined inside eliminates, reduces and/or ameliorates the odor of any fabric or product that is placed in the bag that may contain bacteria on it. The bag also optionally contains a hook or similar device that is associated with the bag allowing the bag to be hung up. For example, the bag can be hung up in a locker.

The bag and other products of the present invention can be used for a plurality of purposes but one purpose that they are not intended for is medical applications such as wound care. Wound care treatment with silver containing bandages generally means that the silver containing bandage is applied to the wound and is not removed until the bandage is to be discarded. In contrast, the bag and other products of the present invention are intended to be reused a plurality of time regardless of the activity wherein their use is employed. In wound care, the silver-containing bandage is not to be removed and reused because dirt and other particles may attach to the silver exacerbating infections that may be associated with a wound (irrespective of the presence of the silver). Rather, the purpose of the bag is to ameliorate the odor causing bacteria for the purpose of reducing odor in the surrounding environment. Accordingly, the bag can be used a plurality of times. In addition, the bag will often be put inside another bag or luggage and will protect the surrounding apparel or gear from the wet, and or smelly gear placed inside the silver-lined bag.

As an example, several tests were carried out with socks that had been used in sporting activities. One of each, still damp sweaty socks, were placed in the silver lined bag and the other in a zip-lock bag. After three days each bag was opened and the sock was removed. The sock that was placed in the zip-lock bag was extremely smelly as the odor causing bacteria continued to grow in the moist environment. In addition, the inside of the zip-lock bag was also extremely pungent, almost to the point of being rancid. The sock that was placed in the silver-lined bag was not very smelly, and the tests showed that the silver lining in the bag not only contained the smell of the sock, but also reduced the smell of the sock. When examined, the inside of the silver-lined bag had a very low residual smell from the sock. The sock that had been placed in the silver-lined bag had only 35% of the intensity of smell of the sock that had been placed in the zip-lock bag.

Due to the strength of the polyurethane/silver coated fabric laminate it can be expected that the bag will last for a long time, and in the normal course of use will last longer than most sports bags or luggage due to its durable design. In addition, the bag is designed for multiple uses and as such can be washed should the outside and/or inside of the bag become dirty.

The bag is of any size so that it can be used for its intended purpose. In one embodiment, the bag is of a size that allows one to place one or more garments in the bag. For example, in one embodiment, the bag is of a size that allows one to place the, soccer shirts and shorts from an entire soccer team. Alternatively, the bag may be of a size that is ideally suited for the sports garments from an individual. Accordingly, the size of the inside of the bag may be ¼ or ½ a cubic foot. Alternatively, the size of the inside of the bag may be 1 cubic foot, or alternatively, the size of the inside of the bag may be 2 or 3 or 4 or 5 a cubic feet or any size between a ¼ cubic foot to 10 cubic feet.

In an embodiment, the bag can be made of a three layer laminate material. The outside layer provides the color and texture and, in one variation, is made of nylon. The nylon can be dyed to add color to it, or printed. The bag optionally also has Velcro and/or magnets associated with the outer layer of the bag so that it can be attached to Velcro and or a metal surface. This allows the bag to be attached (such as being elevated off the ground) by another Velcro strip/pad and/or metal.

The middle layer can be a polyurethane film or another suitable film such as polyethylene, polypropylene, polyetherimide, polyetherimide esters, acrylic resins, synthetic rubbers, ethylene vinyl acetate copolymer resins or other copolymer resins that provides the waterproofing, and additional strength to the bag. In one embodiment, the middle layer is polyurethane. The first layer of the fabric, in one embodiment, is a non-woven 70/30 poly/silver coated nylon blend. Alternatively, the first layer is a 50% polyester and 50% nylon blend, a 30/70 poly/silver coated nylon blend, or a blend between 95/5 and 5/95 poly/silver coated nylon blend. The nylon fibers are electrolessly coated with silver, and by weight, can have a silver coating of as low as 5% and as high as 30%. In another embodiment, the first layer can be comprised of 100% silver coated nylon fabric, knitted, woven or non-woven. The electrolessly coated nylon fibers are coated by silver, and by weight, can have a silver coating as low as 5% and as high as 30% or alternatively, as low as 2% and as high as 30% by weight. In another embodiment, the present invention relates to a foam pad that comprises a three layer laminate having a polyester/silver coated nylon non-woven fabric layer, that is laminated to an open cell foam layer (as the middle layer) and a PSA (pressure sensitive peelable adhesive layer) on the other side. In one embodiment, polyester/silver coated nylon non-woven fabric layer is a non-woven 70/30 poly/silver coated nylon blend. Alternatively, the polyester/silver coated nylon non-woven fabric layer is a 50% polyester and 50% nylon blend, a 30/70 poly/silver coated nylon blend, or a blend between 95/5 and 595 silver coated nylon fibers. In another embodiment, the Polyester/Silver Coated Nylon Non-Woven fabric layer could be a knitted or woven fabric. In addition, the knitted, woven or non-woven silver coated nylon fabric could comprise of 100% silver coated nylon fibers or yarns. The silver coating could be, by weight, as low as 5% of the nylon fibers and up to 30% of the nylon fibers or alternatively, as low as 2% and as high as 30% by weight. The adhesive may be a reactive or non-reactive adhesive. In an embodiment, the adhesive may be either a solvent based adhesive or polymer dispersion adhesives (an emulsion adhesive) or a combination of the two. The adhesive may be one or more of various types of glue, such as a pressure sensitive adhesive or a heat sensitive adhesive. Alternatively and or additionally, the adhesive may be an animal protein based adhesive such as albumin glues, casein glues, or meat glues. The adhesive may be a plant based glue such as Canada balsam, Coccoina, gum Arabic, a latex based glue, a starch based glue, a methyl cellulose based glue, a mucilage based glue, a resorcinol resin, or a urea-formaldehyde based glue. Alternatively and/or additionally, the adhesive may be a polystyrene cement/butanone, or a dichloromethane based adhesive. Alternatively and/or additionally, the adhesive may be an acrylonitrile based glue, cyanoacrylate based glue, an acrylic based monomer glue, or a resorcinol based glue. Alternatively and/or additionally, the adhesive may be an epoxy resin, an epoxy putty, an ethylene-vinyl acetate based glue (hot melt glue), a phenol formaldehyde based glue, a polyamide based glue, a polyester resin based adhesive, a polypropylene based glue, a polysulfide based glue, a polyurethane based glue, a polyvinyl acetate based glue, a polyvinyl alcohol based glue, polyvinyl chloride based glue, a polyvinylpyrrolidone based glue, a rubber cement, a silicone based glue, an acrylic copolymer, a styrene acrylic copolymer based glue, a styrene-butadiene-styrene, a styrene-isoprene-styrene, and/or a silicone polymer based glue. In one embodiment, the foam pad is part of protective gear for work related purposes, such as any profession that uses protective gear including but not limited to construction workers, carpenters, masons, tile layers, factory workers, welders, policemen, firemen, smelt workers, utility workers, and other professions that use protective gear. In another embodiment, the foam pad is part of protective gear for sports related activities. For example, the adhesive, side of the pad may be attached to the protective gear. For example, the protective gear may be used for padding used in any sport including but not limited to football, soccer, lacrosse, hockey, field hockey, fencing, epee, jai alai, sailing, spelunking, diving, scubaing, rowing, basketball, baseball, tennis, cycling, boxing, mixed martial arts, judo, taekwondo, wrestling, dancing, golf, auto racing, volleyball, weightlifting, track and field events, cricket, rugby, horse racing, equestrian events, swimming, polo, water polo, figure skating, speed skating, skiing, ski-jumping, luge, bobsled, skeleton, snowboarding, Olympic game sports, X-games sports, and others. In one embodiment, the foam pad is attached to the protective gear so that the silver side of the foam pad is adjacent to the athlete's skin. Thus, the odor emanating from the bacteria that build up due to sweat is eliminated, reduced and/or ameliorated. In an embodiment, the foam pad is part of or can be adhered to (by the adhesive) to any one or more of a shin pad, a helmet, a shoulder pad, a knee pad, a glove, a chest pad, a sock, a shoe, an arm pad, a leg pad, a hip pad, a back pad, a stomach pad, an elbow pad, an ankle pad, or another pad. In a variation, the foam pad is part of a shin pad, a helmet, a shoulder pad, a knee pad, a glove, a chest pad, a sock, a shoe, a hip pad, an elbow pad, or an ankle pad. In an embodiment, it is part of a shin pad. In another embodiment, the present invention relates to a pad that comprises a two layer laminate comprising a polyester/silver coated nylon non-woven fabric that is laminated to a PSA (pressure sensitive peelable adhesive layer) on one side and a poly/silver nylon on the other side. Similar to the three layer pad, the pad may be associated with protective gear for work related or sports related activities. For example, the adhesive side of the pad may be attached to the protective gear. The adhesive side may use any of the adhesives mentioned above that may be used in connection with the three layered pad and the silver side may be any of the silver ratios that are discussed above in connection with the three layered pad. The silver side is adjacent to the skin of the worker and/or athlete, which when said individual sweats, the odor emanating from the bacteria is eliminated, reduced and/or ameliorated.

In an embodiment, the pad may be part of a mat that can be used for any of a variety of purposes. In one variation, the mat may be designed for young children in school that take naps during their time in school. Alternatively and/or additionally, the pad may be part of a yoga mat or dance mat or a workout mat so that the silver is adjacent to the person working out (or performing yoga or dance). The silver serves the function of eliminating, reducing and/or ameliorating any odor that emanates from the sweat of the person working out and/or performing dance and/or yoga. In an embodiment, the silver may be on the other side (facing the floor) thus killing any bacteria on the floor, which in turn eliminates, ameliorates or reduces the odor emanating from any bacteria. In a variation, the mat that contains silver coated nylon non-woven fabric on one side can be rolled up for easy transportation. In an embodiment, the mat may have foam associated with it for the comfort of the person working out (or for the practitioner of yoga and/or dance). The mat may also have a surface opposite the silver side that has some friction associated with it so that it does not slide on the floor or surface upon which the mat rests when the person sleeps, works out (or performs yoga and/or dance).

In an embodiment, the pad of the present invention can be used as an exercise and/or wrestling mat. In one embodiment, the silver may be present on the other side of where the exerciser and/or wrestler exercises and/or wrestles. When the silver is present on the bottom of the mat, the silver kills any bacteria that may be present on the floor, thus eliminating, ameliorating or reducing any odor emanating from any bacteria that may be on the floor. In a variation, the pad may be part of a wrestling or exercise mat wherein the mat can be rolled up. The silver on one side (or alternatively on both sides) eliminates, ameliorates or reduces the odor emanating from any bacteria when rolled up, greatly reducing any odor.

In an embodiment, the pad may have an adhesive such as Velcro which allows attachment to the top tube or bottom tube of a bicycle (either a road bike or a mountain bike) frame or allows the pad to attach to itself so that a rider on a trainer (or on rollers) may ride in a stationary fashion so that sweat does not drop on the top tube or bottom tube of the bicycle frame. In a variation, the silver is deposed so that it is facing outwards from the bicycle top tube or bottom tube of the frame so that any sweat that hits the pad does not hit the frame thereby eliminating, reducing and/or ameliorating any odor that emanates from the sweat while at the same time protecting the frame from sweat.

In an embodiment, the pads (and other products) are made to absorb a sufficient amount of moisture. In an embodiment, the pads (and other products) have the ability to absorb up to 80% by weight of water within 30 minutes. Alternatively, the pads (and other products) can absorb between 70-105% by weight of water within 30 minutes. In a variation, the pads (and other products) have the ability to absorb up to 50% by weight of water within 30 minutes. In an embodiment, the silver coated non-woven fabric with a foam backing has the ability to absorb up to 300% by weight of water within 30 minutes. The ability of the pads (and other products) to absorb moisture allow sweat to be removed from adjacent the skin (via wicking or some other process) while the silver is still present allowing the pads (and other products) to kill any bacteria that may be present in the sweat, thus eliminating, ameliorating or reducing any odor emanating from any bacteria.

In all embodiments of the silver coated nylon fiber pads, they are not intended for use in wound care as it is expected that the product the pad will be applied to will be reused over and over again after each activity. In normal situations, the odor, on sports or work gear, caused by bacteria, is produced after the wearer has stopped wearing the equipment, and while the gear is still wet. The purpose of applying the pad to sports/work gear and equipment, is so that while the pad is not being worn, which is the majority of the time, the silver has time to kill the odor causing bacteria, thus the next time the activity takes place, there is no odor on the equipment and it can be used again as normal.

### DESCRIPTION OF THE PREFERRED EMBODIMENTS

Examples of products within the scope of the present invention are set forth below.

Silver coated nylon and another non-coated fiber (e.g. polyester), were warp knitted together so that the nylon fibers were on one side of the fabric and the polyester fibers were on the other. The fabric was electrolessly silver coated and the result was a fabric that was silver coated on one side, for example on the nylon fiber side, and the other side was untouched by the silver and retained its color for dying at a later stage. An additional example shows that silver coated yarn can be knit with non-silver coated yarn so that the silver yarn is predominantly on one side of the fabric and the other side is comprised of predominantly the non-silver coated yarn, which can be dyed after the garment has been made.

### Example 1

A warp knit fabric comprised of polyester filament yarn on one side and nylon filament yarn on the other was constructed. The double-sided fabric construction was used so that the polyester showed predominately on one side of the fabric and the nylon yarn showed predominantly on the other side of the fabric. The fabric weight was approximately 3.6 ounces per square yard before the silver plating process and comprised by weight of approximately 50% polyester and 50% nylon. A fabric was used, that had already been disperse dyed, prior to electrolessly silver plating the nylon in the fabric. The dyed fabric was processed using an electrolessly silver plating process that deposited approximately 17% silver onto the nylon fibers. Prior to electrolessly plating the fabric, the garment was scoured and alien chemically treated to sensitize the nylon fibers and enhance the plating process. The fabric was washed thoroughly after the plating to remove any residual chemicals from the process. Once washed, the fabric was dried and rolled onto a tube for further processing.

This fabric was cut and sewn into tee shirts, suitable for athletes. The tee-shirts were cut in such a manner that allowed the dyed side of the double sided fabric to be on the outside of the garment and the silver coated nylon side to be on the inside of the garment.

In another embodiment, this fabric type can be used as a balaclava. The balaclava can be constructed so that the silver coated nylon is on the inside of the garment and the dyed side is on the outside. The silver coated fabric conducts heat, aids in moisture evaporation as the silver very quickly gets to body temperature, and eliminates the odor caused by the bacteria as the silver coated nylon kills the bacteria. This embodiment is useful for motorcyclists as the lower part of the thermally conductive garment would be outside of the motorcycle helmet and would keep the wearer cooler in the summertime.

### Example 2

An alternative method of production was also employed for another garment. A seamless tank top garment was produced using two types of yarn. One yarn, a 70 denier nylon yarn, was knitted to the outside of the garment. The other yarn was a covered yarn comprised of 30 denier silver coated nylon yarn which was wrapped around a core yarn of 20 denier spandex. This covered yarn was knitted on the inside of the garment. Once the garment was knitted into a blank it was cut and seamed to produce the final garment. The garment was dyed and due to the knitting process employed, the silver coated nylon covered yarn was predominately on the inside of the garment. This method of manufacture produced a garment that has the silver on the inside of the fabric, or in other words against the wearer's skin. The garment was then dyed to the desired color. The durable silver coated yarn not only retains its integrity over the target fabric surface, but also continues to provide effective antimicrobial treatment, thermal properties and electrical conductive properties as well. In another version, the fabric could be constructed in the shape of a skull cap for wearers of wigs. The skull cap would make the wearer of the wig feel more comfortable as the silver sided fabric which would be against the skin would be thermally conductive. In addition, the silver would eliminate the body odors that occur as the silver sided fabric would kill the bacteria causing the odor. The two sided fabric allows the non-coated fabric to be dyed to the color of the wig or to the color of the scalp, thus making the wearer feel more comfortable using a skull cap under the wig as it would reduce the conspicuousness of the wig. In one embodiment, the same skull cap can be used for those wearing any type of hats or helmets, for the purposes of moving heat around the skull and eliminating body odors under the hat or helmet. In another embodiment, a glove can be constructed with silver coated nylon on the inside and an alternate dyeable non-silver coated outside. In another embodiment of the glove, the glove can have a glue applied that would allow the glove to be permanently fixed to the inside of any glove so that it in essence the glove becomes a liner for another glove.

### Example 3

In making a bag as discussed above, the process involves coating the non-woven fabric with silver. The poly/nylon fabric undergoes an electroless plating process which results in the nylon fibers being coated with silver. The coating amount by weight is 9%, but can be coated in amounts as low as 5% and as high as 30% or any value in between. In one variation, the amount of silver that is added may be determined by the economics and the cost of silver. After coating the non-woven nylon with silver, the non-woven fabric is now ready to laminate.

Using a combination of glue and heat, the laminator laminates the silver coated non-woven fabric to the middle layer, which is made from polyurethane. Once the two layer laminate has “set-up” the outer layer of nylon fabric that can be dyed is, laminated to the polyethylene, again using glue and heat.

The laminated fabric is the basis for constructing the bag. The bag is designed so the silver coated nylon non-woven fabric is on the inside of the bag and the nylon woven fabric faces the outside although it should be understood that the silver coated fiber may be placed on the outside of the bag, or alternatively on both sides of the bag, particularly in the instance where the bag may be reversible.

The outside fabric can be constructed of many different types of material, including, but not limited to cotton, polyester, nylon, rayon, polypropylene and any combination of these fibers. The outside fabric can be woven, circular knitted, warp knitted or non-woven. In one embodiment, the outside fabric can be dyed. In an embodiment, the outside fabric can be a dyed fabric, a printed vinyl film, or a colored vinyl film or any combination of these.

The laminate can be constructed of another type of film, but should be waterproof and have the ability to adhere to the outer and inner layer.

The inner layer can be constructed of a woven, knit, warp knit or non-woven fabric, with a minimum of 5% of the fibers by weight being silver coated nylon. The yarns in the fabric can be spun or filament but by weight once the fabric is constructed the fabric should contain a minimum of 5% silver coated nylon. This is the amount of silver coated nylon that gives properties sufficient to eliminate, reduce or ameliorate the odor emanating from bacteria.

Heretofore, the inventors are unaware of any one that has used the unique combination of fabrics and films in the construction of this bag.

### Example 4

The pad comprising a three layer laminate material can be made in a process that is described below. The inner layer contains a polyester/silver coated non-woven fabric that provides the anti-microbial power is often adjacent to the skin. The silver coated layer is surprisingly comfortable when adjacent to the skin. This layer of the pad in an embodiment comprises a non-woven 70/30 poly/silver coated nylon blend. The middle layer is an open cell foam that can absorb moisture/sweat. It can be designed so that it absorbs great quantities of moisture (e.g., on the order of hundreds of mg of moisture per gram foam or more). The middle layer, in an embodiment, is designed in such a way that the bacteria from moisture/sweat remains close to the adjacent silver containing layer helping to eliminate, reduce, or ameliorate smell. In one embodiment, wicking of the moisture occurs away from the silver layer but the bacteria remains adjacent to the silver containing layer. In an embodiment, the foam containing layer may also contain an antibiotic (other than silver) that further aids in killing bacteria. This antibiotic may be uniformly spread throughout the foam, or alternatively, there may be a graduated amount of antibiotic with higher levels near the silver layer (or vice versa). The outer layer in an embodiment is a PSA (pressure sensitive peelable adhesive layer), which allows the pad to adhere to a variety of surfaces. In an embodiment, a protective sheet over the adhesive can be peeled off revealing the adhesive, which can then be administered to another surface so that the foam pad adheres. Alternatively, the outer layer may not have a protective sheet but rather the adhesive may be in a form that has to be activated so that it adheres to a surface. Activation may take place by any number of means such as by addition of a solvent to the adhesive, the use of heat so that it activates the adhesive, the use of pressure to activate the adhesive, the combination of these methods, or by some other means.

The process involves coating the non-woven fabric by an electroless plating process which results in the nylon fibers being coated with silver. In one process, the coating of silver on the nylon fibers is such that 9% by weight of silver is added. However, the process can accommodate any level of silver. Generally, the amount of silver tends to be between about 5% and 30% by weight. After the non-woven fabric has been coated with silver, this layer is now ready to laminate. Using a combination of glue and heat, the laminator laminates the silver coated non-woven fabric to the foam layer. Once the two layer laminate has “set-up” the outer layer of PSA is laminated to the foam, again using glue and heat.

The pad is designed so that the silver coated nylon non-woven fabric is on the outside of a protective pad and is easily applied to a shin guard or other type of protective product that comes in contact with sweat using the peelable sticky backing that has been applied. The foam can absorb excess moisture and keep moisture off the skin. As the moisture passes through the silver coated nylon fabric it picks up silver ions, causing the odor causing bacteria to die.

In an embodiment, the shin guards, pads (and other products) are made to absorb a sufficient amount of moisture. In an embodiment, the pads (and other products) have the ability to absorb up to 80% by weight of water within 30 minutes. Alternatively, the pads (and other products) can absorb between 70-105% by weight of water within 30 minutes. In a variation, the pads (and other products) have the ability to absorb up to 50% by weight of water within 30 minutes. The ability of the shin guards, pads (and other products) to absorb moisture allow sweat to be removed from adjacent the skin (via wicking or some other process) while the silver is still present allowing the shin guards, pads (and other products) to kill any bacteria that may be present in the sweat, thus eliminating, ameliorating or reducing any odor emanating from any bacteria.

In an embodiment, the shin guard (or other protective padding) has at least one side of the shin guard (or protective padding) that is sufficient to protect the wearer from harm. In one embodiment, the shin guard (or protective padding) is able to withstand blows comparable in order to pass the tests as enumerated in prEN 13061 (Protective Clothing—Shin Guards For Association Football Players—Requirements And Test Methods), in ASTM 1446 (Standard Test Methods For Equipment And Procedures Used In Evaluating The Performance Of Protective Headgear), or in Technical report J211 A (“Instrumentation For Impact Tests—SAE J211A”. Society For Automotive Engineers, Inc. 1973 SAE Handbook). The shin guard (or other protective padding) are of a size that is able to sufficiently protect the area that one desires to be protected as enumerated in the document Test Method And Standard Performance Specification For Newly Manufactured Soccer Shin Guards prepared by NOCSAE (National Operating Committee On Standards For Athletic Equipment) DOC (ND) 090-06m07, which is hereby incorporated by reference in its entirety. This document is also incorporated by reference in its entirety for all other purposes.

These pads also in one embodiment have a primary protective component of the shin guard, which provides the impact resistance, which is not impacted by the pad. In one embodiment, the pad makes the shin guard more comfortable to wear as the foam assists in keeping the shin guard in place. Moreover, the foam in one embodiment helps the shin guard conform to the contours of the shin.

These pads also have the absorption capabilities as discussed above, for example, the silver coated non-woven fabric with a foam backing has the ability to absorb up to 300% by weight of water within 30 minutes.

In another embodiment, a pad has been developed that will eliminate odors from shoes. In one variation, the pad is added to the outside of a wooden or plastic shoe tree. Plastic shoe trees are readily available as there are many manufacturers of these blow molded type products. In one variation, the shoe odor eliminating product has been developed by constructing a Sock made up of 5-50% silver coated nylon and the remainder is one or more synthetic filament or spun yarns which absorb the moisture, such as Sorbtek® polyester textured yarn (UNIFI, North Carolina). No natural fibers can be used in the Sock. In one embodiment, the Sock can be placed over a shoe tree and the sock sewn closed, permanently by sewing, or by some other method such as the use of Velcro. The shoe tree, with the silver Sock over the shoe tree, can then be inserted in a shoe to absorb the moisture and kill the odor causing bacteria, thereby eliminating, ameliorating or reducing any odor being created by bacteria. In one embodiment, after the Sock has been placed over the shoe tree and closed, a loop of string or narrow fabric may be added around the heel area of the shoe tree to allow the shoe tree to be more easily removed from the shoe.

In another embodiment, a saddle pad (for riding horses) can be constructed by stitching the silver coated nylon fabric onto the outside of a saddle pad. The silver coated nylon fabric can be stitched to one side only or alternatively, to both sides, replacing the existing outer layer of cotton or other non-silver coated fabric, stitched to the outside layer directly. In one embodiment, the non-woven fabric can be used to reduce the slippage of the saddle pad on the horses back or against the saddle, but in an alternative embodiment the silver coated fabric can be woven or knitted. By adding the silver coated nylon fabric to the saddle pad, sweat is absorbed and the sweat conies in contact with the silver. The silver kills the bacteria in the sweat of the animal that causes the saddle pad to smell, thus eliminating or at the very least significantly reducing the smell of the saddle pad. In an another embodiment, the saddle pad may comprise a three part laminate that is a part of the saddle pad. The outer layer may be made of a non-woven silver coated fabric, the inner layer may be an open cell foam, which facilitates greater absorption of sweat, and the inner layer in one embodiment comprises a peelable sticky back. By removing the backing, the pad can be applied to any shape or type of existing saddle pad.

The silver coated nylon fabric can be constructed of many different combinations of fibers, both filament and spun, including, cotton, polyester, nylon, rayon, polypropylene and any combination of these fibers.

In addition, the silver coated fabric can be constructed of a woven, knit, warp knit or non-woven fabric. Generally, 5% or more of the fibers by weight are silver coated nylon. The yarns in the fabric can be spun or filament but by weight once the fabric is constructed the fabric to be an effective bacterial killer should contain about at least 5% silver coated nylon.

Heretofore, the inventors are unaware of any one that has used the unique combination of fabrics, foam and PSA in the construction of pads.

### Example 5

In an embodiment, a pad comprising a two layer laminate material is designed and made as described below. The outside layer of the pad contains polyester/silver coated non-woven fabric that provides the anti-microbial effect. As described above, the silver containing side when against the skin is surprisingly comfortable. In an embodiment, the outside layer of the pad is a non-woven 70/30 poly/silver coated nylon blend but other ratios are possible such as any level between about 95/5 and 10070/30 poly/silver coated nylon blend.

In an embodiment, the outer layer is a PSA (pressure sensitive peelable adhesive layer) on the outside which allows the pad to adhere to a variety of surfaces. In an embodiment, a protective sheet over the adhesive can be peeled off revealing the adhesive, which can then be administered to another surface so that the foam pad adheres. Alternatively, the outer layer may not have a protective sheet but rather the adhesive may be in a form that has to be activated so that it adheres to a surface. Activation may take place by any number of means such as by addition of a solvent to the adhesive, the use of heat so that it activates the adhesive, the use of pressure to activate the adhesive, the combination of these methods, or by some other means.

One process that can be used to coat the non-woven fabric with silver comprises undergoing an electroless plating process which results in the nylon fibers being coated with silver. In one embodiment, the level of coating by weight is 9%, but levels as low as 5% and as high as 30% can be used with one factor that can determine the amount of silver being used is the cost of silver. Alternatively, the cost may be based upon the desired kill rate of bacteria (e.g. generally bacteria kill rates are optimized at amounts above about 5%). After addition of the silver, the non-woven fabric is now ready to laminate.

Using a combination of glue and heat, the laminator laminates the silver coated non-woven fabric to the layer of PSE using glue and heat.

The pad is designed so the silver coated nylon non-woven fabric is on the outside of a protective pad and is easily applied to a shin guard or other type of protective product that comes in contact with sweat. The peelable sticky backing can be applied to the protective gear. The different types of protective gear to which the pad can be administered are discussed above.

The silver coated fabric can be constructed of many different combinations of fibers, both filament and spun, including, cotton, polyester, nylon, rayon, polypropylene and any combination of these fibers.

In addition, the silver coated fabric can be constructed of a woven, knit, warp knit or non-woven fabric. In an embodiment, a minimum of 5% of the fibers by weight are silver coated nylon. The yarns in the fabric can be spun or filament but by weight once the fabric is constructed the fabric generally should contain amounts that are at least about 5% silver coated nylon.

Heretofore, the inventors are unaware of any one laminating a silver coated fabric laminated to a PSA and the method of construction of said two layer pad as discussed above.

Accordingly, in one embodiment, the invention relates to a two sided fabric that comprises a first side and a second side, wherein the first side comprises nylon fibers coated with silver and the second side being a non-nylon fiber that has not been coated with silver. In a variation, three or more fiber types can be used in one fabric.

In a variation of this embodiment, the second side is a filament polyester yarn or a spun polyester yarn or spun polyester and cotton yarn blend.

In a further variation, the first side cannot be dyed with color using normal dye procedures. By normal dye procedures it is meant disperse dyeing and/or acid dyeing. In a further variation, the second side can be dyed using normal dye procedures.

In an embodiment, the fabric is very durable so that silver is integrally retained with the nylon fibers so that after 30 washes, as performed in accordance with wash procedure of AATCC test method 130-1981, at least 65% of the silver on the first side is retained relative to an amount of silver on the first side prior to any wash. In a variation, at least 75% of the silver on the first side is retained relative to an amount of silver on the first side prior to any wash under the same conditions. In a further variation, at least 85% of the silver on the first side is retained relative to an amount of silver on the first side prior to any wash.

In an alternate embodiment, the first side with the silver coated fiber is electrically conductive. In a variation of this embodiment, the silver is present in an amount that allows between 1 amp of current at 24 volts to 100 amps of current at 24 volts over one square meter of fabric. In a variation, the silver may be present in an amount that allows between 10 amps of current at 24 volts to 75 amps of current at 24 volts over one square meter of fabric. The silver may be present in parallel of in series to attain this current at the specified voltage.

In one embodiment, the silver on said first side is applied electrolessly. In a variation, the silver may be applied electrolytically.

In a variation of an embodiment, the said first side of the fabric is thermally more conductive than the second side.

In one embodiment, the fabric on one side is 100% nylon fabric.

In an alternate embodiment, the invention relates to a method of manufacturing a two sided fabric that is silver coated on a first side and not silver coated on a second side, wherein the method comprises using a method selected from the group consisting of a non-woven method, a warp knit method, a circular knit method and a woven method to produce a base fabric that has a nylon fiber on the first side and a non-nylon fiber on the second side wherein the base fabric is then processed by electrolessly silver plating the base fabric so that to produce the two sided fabric that has nylon fibers coated with silver on the first side and non-nylon fibers without a silver coating on the second side.

In a variation of the method, the base fabric can be dyed prior to the electrolessly silver plating step. Alternatively, the fabric can also be dyed if an electrolytic silver plating step is used.

In an embodiment, the first side of the two sided fabric exhibits a log kill rate for Staphylococcus aureus of at least 1.5 and a log kill rate for Klebsiella pneumoniae of at least 1.5 after 30 washes as performed in accordance with wash procedure AATCC Test Method 130-1981.

In an embodiment, the first side has between about 8% by weight silver and 35% by weight silver. Alternatively, the first side has between about 15 and 25% by weight silver, or alternatively between about 18 and 22% silver.

The fabric of the invention can be used for any of a plurality of items. For example, the fabric can be used in any athletic gear, including tee-shirts, shorts and sweatpants. It is also easily applicable to pants or dresses, for socks, for nightwear such as nightgowns and/or pajamas, for bed sheets and/or pillowcases, for shorts, for underwear, for undergarments such as bras, or the fabric can be used in any of a plurality of other items. The fabric can also be used as a medical garment.

In an alternate embodiment, the fabric can be used to alleviate symptoms from shingles. In a variation, the method of alleviating pain symptoms from shingles in an individual comprises wearing an article of clothing comprising a two sided fabric with an inside and an outside, the inside of the fabric contacts the individual and comprises a nylon fiber coated with silver, the outside does not contact the individual and comprises a non-nylon fiber without a silver coating.

In an embodiment, the present invention relates to a bag that comprises three layers, a first layer, a second layer and a third layer, the first layer laminated together with the second layer and the second layer laminated to the third layer, wherein the inside of the bag comprises the first layer comprising a non-woven nylon that has been at least partially coated with silver, the second layer comprising a waterproof layer that adds structural stability to the bag, and the third layer comprising a dyeable layer that is made from one or more members selected from the group consisting of cotton, polyester, nylon, rayon, polypropylene and combinations thereof.

In an embodiment, the bag may have at least one of the first layer laminated together with the second layer and the second layer laminated to the third layer by use of an adhesive. Alternatively, both may be laminated by use of an adhesive. In a variation, the adhesive may be applied so that adhesion occurs by the use of a combination of pressure and heat. Alternatively, either of pressure or heat may be used.

In an embodiment, the adhesive may be one or more members selected from the group consisting of an acrylic copolymer, a styrene acrylic copolymer based glue, a styrene-butadiene-styrene, a styrene-isoprene-styrene, and a silicone polymer based glue or combinations thereof. Other adhesives as described herein can also be used.

In a variation, the amount of silver is between about 5% and 30% silver by weight, between about 7 and 25%, between about 7 and 20% between about 7 and 12%, between about 8 and 10% and about 9% silver by weight.

In one embodiment, the bag comprises a first layer that comprises silver coated nylon fibers and/or silver coated nylon yarns and optionally also contains polyester or other non-nylon synthetic fibers or non-nylon synthetic yarns, wherein the silver coated nylon fibers and/or the silver coated nylon yarns in the first layer are by weight between about 5% and 100% of the first layer, the second layer is polyurethane, polyethylene or polypropylene, and the third layer is cotton, polyester, nylon, rayon, polypropylene or combinations thereof.

In a variation, the second layer is polyurethane, polyethylene or polypropylene and the third layer is cotton, polyester, nylon, rayon, polypropylene or combinations thereof. Alternatively, the second layer is polyethylene and the third layer is cotton, polyester, nylon, rayon, polypropylene or combinations thereof. Alternatively, the second layer is polypropylene and the third layer is cotton, polyester, nylon, rayon, polypropylene or combinations thereof. Alternatively, the second layer is polyethylene and the third layer is cotton, polyester, nylon, rayon, or polypropylene. Alternatively, the second layer is polypropylene and the third layer is cotton, polyester, nylon, rayon, or polypropylene. Alternatively, the second layer is polyethylene and the third layer is cotton, polyester, nylon, or rayon, or the second layer is polyethylene and the third layer is cotton. Alternatively, the second layer is polyethylene and the third layer is polyester, nylon, or rayon, or the second layer is polyethylene and the third layer is nylon. Alternatively, the second layer is polyethylene and the third layer is cotton or polyester, nylon, or rayon, or the second layer is polyethylene and the third layer is cotton, or the second layer is polyethylene and the third layer is nylon.

In an embodiment, the bag is constructed as a sheet and the sheet is sewed and/or laminated together. In a variation, when the bag is made as a sheet, the bag can be folded in half (with the silver on the inside) and the first side can be laminated to the first side to generate a bag. Alternatively, when the bag is made as a sheet, the bag can be folded in half (with the silver on the inside) and the folded sheet can be sewed or stitched together to generate a bag. Alternatively, the bag can be constructed in a manner that is seamless. In an embodiment, the bag may further comprise a zipper and a hook. Alternatively, the bag may contain a zipper or a hook. The hook can be any of a number of types of hooks that allow the bag to be hung up.

In an embodiment, the present invention relates to methods of eliminating, reducing or ameliorating smells and/or odors from odor causing bacteria. In an embodiment, the silver may be present on any of the products described herein. In a variation, the method of ameliorating, reducing and/or eliminating the smell results from the release of silver ions upon activation with moisture so that they kill bacteria, thereby eliminating, reducing and/or ameliorating smell from the bacteria. In an embodiment, the present invention relates to a method of ameliorating or reducing odors for one or more garments comprising placing the one or more garments in a bag, wherein the bag comprises three layers, a first layer, a second layer and a third layer, the first layer laminated together with the second layer and the second layer laminated to the third layer, wherein the inside of the bag comprises the first layer comprising a non-woven nylon that has been coated with silver, the second layer comprising a waterproof layer that adds structural stability to the bag, and the third layer comprising a dyeable layer that one or more members selected from the group consisting of cotton, polyester, nylon, rayon, polypropylene and combinations thereof. In an alternate embodiment, any odor resulting from a wearer wearing a garment(s) may be eliminated, reduced and/or ameliorated simply by wearing the garment.

In an embodiment, if the bag is used to reduce, eliminate and/or ameliorate the smell, the bag may contain a zipper and the method further comprises zipping up the bag so that the bag is closed (i.e., the inside cannot be seen without unzipping the bag).

In an embodiment, the present invention relates to protective gear that comprises a foam pad, wherein the foam pad comprises two layers, the two layer foam pad comprising a first layer and a second layer, wherein the first layer is a polyester/silver coated nylon non-woven fabric layer, that is laminated to the second layer comprising pressure sensitive peelable adhesive layer; wherein the first layer is between about 70/30 and 30/70 by weight poly/silver coated nylon blend or alternatively between about 85/15 and 15/85 by weight poly/silver coated nylon blend, or alternatively between about 95/5 and 5/95 by weight poly/silver coated nylon blend. In an embodiment, the protective gear further comprises a hard surface (e.g., a hard plastic) that protects a wearer of the protective gear. In a variation, the hard surface is the layer that is the most outside (i.e., the furthest from the skin of the wearer).

In an embodiment, the present invention relates to protective gear that comprises a foam pad, wherein the protective gear comprises a foam pad having three layers, the three layer foam pad comprising a first layer, a second layer and a third layer, wherein the first layer is a polyester/silver coated nylon non-woven fabric layer, that is laminated to the second layer comprising an open cell foam layer that is laminated to a pressure sensitive peelable adhesive layer; wherein the first layer is between about 70/30 and 30/70 by weight poly/silver coated nylon blend.

In an embodiment, the first layer of the protective gear is between about 70/30 and 40/60 by weight poly/silver coated nylon blend. In an embodiment, the first layer of the protective gear is about 70/30 by weight poly/silver coated nylon blend.

In an embodiment, the protective gear further comprises one or more members selected from the group consisting of a shin pad, a helmet, a shoulder pad, a knee pad, a glove, a chest pad, a sock, a shoe, an arm pad, a leg pad, a hip pad, a back pad, a stomach pad, an elbow pad, and an ankle pad. In a variation, the protective gear may be one or more members selected from the group consisting of a shin pad, a helmet, a shoulder pad, a knee pad, a glove, a chest pad, a sock, a shoe, an arm pad, a leg pad, a hip pad, a back pad, a stomach pad, an elbow pad, and an ankle pad. In a variation, the protective gear may be a shin pad. In a variation, the protective gear may contain a spongy layer as the outermost layer after the two or three layer foam pad has been added to the protective gear. This spongy layer may aid the wearer in preventing injury and/or protect the wearer as is necessary.

In a variation, the first layer of the protective gear comprises between about 60/40 to 40/60 poly/silver coated nylon non-woven blend or alternatively between about 85/15 and 15/85 by weight poly/silver coated nylon blend, or alternatively between about 95/5 to 5/95 poly/silver coated nylon non-woven blend.

It is contemplated and therefore within the scope of the invention that any feature that is disclosed herein can be combined with any other feature. When a range is disclosed, it is contemplated and therefore within the scope of the invention that any real or irrational number that fits within that range is contemplated and therefore within the scope of the invention, even if that number is not explicitly mentioned. Minor modifications to the invention are contemplated and also within the scope of the invention. Nevertheless, the invention is defined by the below claims.

We claim:

1. A bag that comprises three layers, a first layer, a second layer and a third layer, the first, layer laminated together with the second layer and the second layer laminated to the third, layer, wherein the inside of the bag comprises the first layer, which comprises a knitted, woven or non-woven fabric that contains silver coated nylon fibers or silver coated nylon yarns, spun yarns or filament yarns, the second layer comprises a waterproof layer that adds structural stability to the bag, and the third layer comprises a dyeable layer that comprises one or more members selected from the group consisting of cotton, polyester, nylon, rayon, polypropylene and combinations thereof.

2. The bag of claim 1, wherein at least one of the first layer laminated together with the second layer or the second layer laminated to the third layer uses an adhesive.

3. The bag of claim 2, wherein the adhesive adheres using a combination of pressure and heat.

4. The bag of claim 3, wherein the adhesive is one or more members selected from the group consisting of an acrylic copolymer, a styrene acrylic copolymer based glue, a styrene-butadiene-styrene, a styrene-isoprene-styrene, a silicone polymer based glue and combinations thereof.

5. The bag of claim 1, wherein the silver coated nylon fibers or yarns in the first layer comprises between about 2% and 30% silver by weight of the nylon.

6. The bag of claim 5, wherein the first layer comprises about 5% to 30% silver by weight.

7. The bag of claim 1, wherein the first layer comprises silver coated nylon fibers and/or silver coated nylon yarns and optionally also contains polyester or other non-nylon synthetic fibers or non-nylon synthetic yarns wherein the silver coated nylon fibers and/or the silver coated nylon yarns in the first layer are by weight at least about 5% of the first layer, the second layer is polyurethane, polyethylene or polypropylene, and the third layer is cotton, polyester, nylon, rayon, polypropylene or combinations thereof.

8. The bag of claim 7, wherein the second layer is polyurethane.

9. The bag of claim 1, wherein the bag is constructed as a sheet and the sheet is sewed and/or laminated together.

10. The bag of claim 1, wherein the bag is constructed in a manner that is seamless.

11. The bag of claim 1, wherein the bag further comprises a zipper and/or a hook.

12. A method of ameliorating or reducing odors for one or more garments comprising placing the one or more garments in a bag, wherein the bag comprises three layers, a first layer, a second layer and a third layer, the first layer laminated together with the second layer and the second layer laminated to the third layer, wherein the inside of the bag comprises the first layer comprising a combination of nylon/other synthetic fibers or filament, wherein the nylon fiber or yarn contained in the first layer has been coated with silver, the second layer comprising a waterproof layer that adds structural stability to the bag, and the third layer comprising a dyeable or printable layer that comprises one or more members selected from the group consisting of cotton, polyester, nylon, rayon, polypropylene and combinations thereof.

13. The method of claim 12, wherein the bag further comprises a zipper or a fabric hook-and-loop fastener and optionally the method further comprises zipping up or sealing the bag.

14. Protective gear that comprises a foam pad, wherein the foam pad comprises two layers, the two layer foam pad comprising a first layer and a second layer, wherein the first layer is a polyester/silver coated nylon non-woven fabric layer, that is laminated to the second layer comprising pressure sensitive peelable adhesive layer; wherein the first layer is between about 95/5 and 5/95 by weight poly/silver coated nylon blend.

15. Protective gear that comprises a foam pad, wherein the protective gear comprises a foam pad having three layers, the three layer foam pad comprising a first layer, a second layer and a third layer, wherein the first layer is a polyester/silver coated nylon non-woven fabric layer, that is laminated to the second layer comprising an open cell foam layer that is laminated to the third layer, which comprises a pressure sensitive peelable adhesive layer.

16. The protective gear of claim 15, wherein the first layer is between about 95/5 and 5/95 by weight polyester/silver coated nylon blend.

17. The protective gear of claim 14, wherein the protective gear is one or more members selected from the group consisting of a shin pad, a shin guard, a helmet, a shoulder pad, a knee pad, a glove, a chest pad, a sock, a shoe, an arm pad, a leg pad, a hip pad, a back pad, a stomach pad, an elbow pad, and an ankle pad.

18. The protective gear of claim 15, wherein the protective gear further comprises one or more members selected from the group consisting of a shin pad, a shin guard, a helmet, a shoulder pad, a knee pad, a glove, a chest pad, a sock, a shoe, an arm pad, a leg pad, a hip pad, a back pad, a stomach pad, an elbow pad, and an ankle pad.

19. The protective gear of claim 17, wherein the protective gear is a shin guard.

20. The protective gear of claim 15, wherein the first layer comprises between about 85/15 to 15/85 polyester/silver non-woven coated nylon blend.
